(19) United States
(12) Patent Application Publication
(10) Pub. No.: US 2025/0025311 A1
(43) Pub. Date: Jan. 23, 2025

(54) LATERAL AND MEDIAL PIVOTING KNEE PROSTHESIS

(71) Applicant: William Andrew Hodge, Tampa, FL (US)

(72) Inventor: William Andrew Hodge, Tampa, FL (US)

(21) Appl. No.: 18/828,412

(22) Filed: Sep. 9, 2024

Related U.S. Application Data
(63) Continuation-in-part of application No. 15/746,200, filed on Jan. 19, 2018, now Pat. No. 12,083,019.
(60) Provisional application No. 62/195,367, filed on Jul. 22, 2015.

(51) Int. Cl. A61F 2/38; A61F 2/30

(52) U.S. Cl. CPC A61F 2/3836; A61F 2/3859; A61F 2/3886; A61F 2/389; A61F 2002/30131; A61F 2002/30245

(57) ABSTRACT

An implantable, asymmetrical right or left knee prosthesis for the arthritic, aging, ligament-deficient knee comprising a femoral component, a tibial component and a patella component, forming three interactive knee compartments mainly the lateral, medial, and femoral patellar compartments, articulating together with a dual, axial pivot knee motion. This dual pivot motion pattern is accomplished by four femoral component radii coacting with four reciprocal proximal tibial surface recesses (in a variety of locations on the tibia based on surgeon choice and patient anatomy), accomplishing initial external femur rotation on the tibia (first pivot location), guiding alignment of the femoral-patellar groove with the patellar quadriceps muscle providing knee strength, durability, and stability. The second pivot point develops with the minor femoral radius in the opposite knee compartment to guide internal rotation of the femur on the tibia to prevent boney impingement of the femur on the tibia and allow deep flexion activities.

### PRIORITY CLAIM

In accordance with 37 C.F.R. 1.76, a claim of priority is included in an Application Data Sheet filed concurrently herewith. Accordingly, the present invention claims priority as a continuation-in-part application of U.S. patent application Ser. No. 15/746,200, entitled “LATERAL AND MEDIAL PIVOTING KNEE PROSTHESIS”, filed on Jan. 19, 2018, which is a § 371 national stage entry of International Application No. PCT/US16/43333, entitled “LATERAL AND MEDIAL PIVOTING KNEE PROSTHESIS”, filed on Jul. 21, 2016, which claims priority to U.S. provisional patent application 62/195,367, filed on Jul. 22, 2015, the entire contents of which are incorporated herein by reference.

### BACKGROUND OF THE INVENTION

Disease, age, and trauma take their toll on the cartilage of the knee to the point where no cartilage remains and pain is experienced due to bone on bone contact.

Knee prosthesis for replacement of a knee joint damaged due to injury or disease generally includes femoral and tibial components to provide new contact surfaces for the joint. These devices try to mimic the motion of a healthy knee and rely on the medial condyle for the primary pivot point.

Koo et al: have shown that the knee moves in a laterally pivoting motion during walking. Koo, Seung bum and Andriacchi, Thomas P., The knee joint center of rotation is predominantly on the lateral side during normal walking, Journal of Biomechanics 41 (2008); 1269-1273.

Kozanek et al, teach that knee kinematics is dependent on load and flexion and that motion of the knee cannot be generalized to a single pattern. Koznanek, Michal et al, Tibiofemoral kinematics and condylar motion during the stance phase of gait, Journal of Biomechanics, 42, (2009); 1877-1884.

Hoshino and Tashman teach sliding in the medial compartment occurs in some movement and corresponds to a lateral pivot at least during running. Hosino, Yuichi and Tashman, Scott, Internal tibial rotation during in vivo, dynamic activity induces greater sliding of tibio-femoral joint contact on the medial compartment, Knee Surg Sports traumatol Arthrosec 20 (2012): 1268-1275.

Banks et al: has shown with Fluoroscopic 3-dimensional imaging, that in the knee with anterior cruciate ligament (ACL) deficiency (99% of total knee replacements are ACL deficient), the motion pivots about the lateral compartment in the first half of the flexion cycle. Also, this motion allows for early engagement of the patella/quadriceps muscle complex providing optimal strength. Banks, Scott A, et al.; Making Sense of Knee Arthroplasty Kinematics: News You Can Use; Journal of Bone & Joint Surgery, Vol. 85A Supplement 4.

Hodge et al. has shown wear patterns in arthritic knees demonstrate a lateral pivoting wear out foot print which has been measured. Similar wear out patterns were seen in traditional total knee arthroplasty (“TKA”). Hodge, W A et al.; Journal of Arthroplasty, Vol 24, Issue 3, pp 448-453 (April 2008).

Banks et al showed the benefits of several pivoting design knee replacements and discussed the reasons for their success. J of Knee Surgery, Vol 32, #7, March 2019.

Meneghini et al in 2024 has shown in a large study of 1306 consecutive knee replacement using a pivoting tibial bearing had better results than most other knee replacement designs. Meneghini et al, J of Arthroplasty, April 2024.

Tuttle et al., U.S. Pat. No. 7,261,740 teach a medial approach to knee replacement in which the medial condyle slides and pivots during extreme motion.

Kauffman et al, U.S. Pat. No. 6,013,103 teaches a medial approach to knee replacement with a knee prosthesis for replacing at least a portion of a knee joint between the distal end of a femur and the proximal end of a tibia. The knee prosthesis includes a tibial component for mounting to the proximal end of the tibia, this tibial component including a proximal surface having a hemispherical medial compartment and a lateral compartment; and a femoral component for mounting to the distal end of the femur, this femoral component including a hemispherical medial condylar portion for pivotally coacting with the hemispherical medial compartment of the tibial component and including a hemispherical lateral condylar portion for movably coacting with the lateral surface of the tibial component; the hemispherical medial condylar portion being substantially congruent with the hemispherical medial compartment of the tibial component so that substantially complete surface-to-surface contact between the hemispherical medial compartment of the tibial component and the hemispherical medial condylar portion of the femoral component is provided throughout the range of flexion of the knee joint.

The prior art approaches to knee replacement try to mimic the motion of a normal healthy knee. The knee joint consists of the distal femur bone with two condyles (medial & lateral) which interact with the tibial bone having a medial and lateral plateau for these condyles to have contact with. There is a third knee compartment consisting of the patella bone riding against the anterior femoral grooved surface called the femoral-patellar compartment which guides the patellar/quadriceps muscle complex which powers the knee and provides stability. These prior approaches however, conveniently ignore the fact that as the body ages and ligaments deteriorate, normal motion is no longer possible.

While the literature suggests the need for incorporation of a “stable” (a guided, controlled, contained, predictable, smooth, regular motion which eliminates slipping, irregular, erratic, and unpredictable motions), pivot points during the flexion cycle, no such designs have been forthcoming. More specifically, nothing in the known prior art discloses or suggests a knee prosthesis including a tibial component for mounting to the proximal end of the tibia, the tibial component including a proximal surface having both hemispherical and curved trough recesses in both medial and lateral compartments allowing for femoral external rotation in early flexion which creates knee power by aligning with the quadriceps mechanism The patient's anatomy and the surgeon's choice dictate whether the initial pivot point is in the medial or lateral compartment. Once the initial stable pivot point is chosen, the second pivot point follows automatically (with high flexion) in the opposite compartment with the minor femoral condylar radius. This creates internal femoral rotation which allows for higher knee flexion activities such as kneeling sitting and squatting.

### BRIEF SUMMARY OF THE INVENTION

Embodiments of the invention comprise an implantable, asymmetrical right or left knee prosthesis for the arthritic, aging, ligament-deficient knee having a femoral component, a tibial component and patella component forming three interactive knee compartments mainly the lateral, medial, and femoral patellar compartments articulating together with a dual, axial pivot type of knee motion. This novel design accomplishes this dual pivot motion pattern with four femoral component radii coacting with four reciprocal proximal tibial surface recesses (in a variety of locations on the tibia based on surgeon choice and patient anatomy) accomplishing initial external femur rotation on the tibia (first pivot location) guiding alignment of the femoral-patellar groove with the patellar quadriceps muscle providing knee strength, durability, and stability. Then, depending where this first pivot point occurs, the second pivot point develops with the minor femoral radius in the opposite knee compartment to guide internal rotation of the femur on the tibia to prevent boney impingement of the femur on the tibia and allow deep flexion activities. This creates two tibial shapes (major medial pivot or major lateral pivot) for each right or left knee based on the surgeon's choice of first (major radii) pivot point location on the tibia implant.

As the human body ages and ligaments deteriorate, normal motion is no longer possible. Embodiments of the invention take into account that the knee being replaced is no longer a normal healthy knee and reengineers the pivot points to accommodate the mechanical realities of an aging body. This means the pivot points of the present invention, which may also be referred to as a dual pivot knee prosthesis, can be artificially varied to accomplish the needed knee mechanics for various knee activities, such as walking, running, stair climbing, kneeling, squatting, and rising from a chair. This design consists of a metal femoral resurfacing (reciprocal right and left knee shapes) each with a medial condyle and a lateral condyle. Each of these femoral condyles have a major radius distally and a minor radius posteriorly. There are two tibial surface recess configurations which can be used for each femoral implant: this first tibial configuration-called ‘Medial Pivot’ has the knee medial femoral condyle (major radius) pivoting on the medial tibial hemispherical recess during walking and running activities; while the second tibial configuration-called ‘Lateral Pivot’ has the lateral femoral condyle (major radius) pivoting with the lateral tibial hemispherical recess in initial knee flexion to accommodate walking and running activities. Both tibial configurations accomplish the same motion between the femur and tibia which aligns the femoral-patellar groove with the patellar/quadriceps muscle to power the high load activities of walking and running.

Both initial stable pivot location, the ‘Medial Pivot’ or the ‘Lateral Pivot’ create external femoral rotation on the tibia and alignment of the patellar/quadriceps mechanism with the femoral-patellar groove, providing optimal knee power during these high load activities of walking and running. Later in the flexion cycle for activities like squatting or rising from a chair, the stable pivot is switched to the opposite compartment of the knee now utilizing the minor femoral condyle radius with the smaller tibial hemispherical recess to then cause internal rotation of the femur on the tibia allowing for higher flexion activities like kneeling, sit-to-stand and squatting. For example, in the ‘Medial Pivot’ tibial configuration, the pivot point switches to the smaller lateral hemispherical recess and for the ‘Lateral Pivot’ tibial configuration, the pivot point switches to the smaller medial hemispherical recess, both creating the internal femoral rotation needed to avoid any boney femoral impingement and allow these high flexion activities.

The knee implant allows for a replaced knee to have two (dual) pivot locations. The first pivot (either ‘Medial Pivot’ or ‘Lateral Pivot’) begins in full knee extension and continues through the first 50% of the flexion cycle guiding femoral external rotation to align the femoral-patellar groove with the patellar quadriceps mechanism providing strength and stability for walking and running. The second pivot (with the smaller, minor femoral radius) follows in the second 50% of the flexion cycle (high flexion) guiding internal femoral rotation which prevents boney impingement of the femur on the tibia during deep flexion activity such as kneeling and squatting. The surgeon chooses the first pivot point as either in the ‘Medial Pivot’ or ‘Lateral Pivot’ and involves one of the distal major radii of the femoral condyle. This pivot point allows for femoral external rotation to align the quadriceps muscle and patella with the femoral intercondylar patellar groove, providing optimal mechanical advantage to the muscle and therefore optimal knee stability and power. Once the first pivot point is chosen, the second pivot point follows automatically in the opposite knee compartment with the minor radius of the femoral condyle to accommodate internal femoral rotation and allow for the higher flexion activities by avoiding femoral boney impingement with the tibia.

Embodiments of the invention provide a right and left knee prosthesis for replacing at least a portion of a knee joint between the distal end of a femur and the proximal end of a tibia which allows for improved movement, stronger function, and lower wear.

Embodiments of the knee prosthesis include, in general, a right and left tibial potentially asymmetrical implant for optimally mounting to the proximal end of the tibia, the tibial component includes medial and lateral sides, each with a hemispherical recess and a curved-trough like recess. The femoral component mounted to the distal end of the femur, includes medial and lateral condyles each with a major (distal) radius and a minor (posterior) radius designed for coacting in a pivoting fashion with the hemispherical recesses of the tibial component. The convex, hemispherical distal femoral condylar portion having stable, pivoting motion (‘Medial Pivot’ or ‘Lateral Pivot’) on the concave (hemispherical) tibial articular surface by having optimal congruency, while the opposite femoral condyle glides in the coronal plane while remaining partially congruent in this plane with the curved half-pipe shaped recess of the tibial component. In knee extension, there is a ball & socket type contact in one compartment where the initial stable pivot point is placed (either medial or lateral), while the other femoral condyle glides smoothly forward or backward in the curved-half pipe tibial recess, providing the external femoral rotation and muscle alignment needed during the first half of the flexion cycle of the knee.

It is an object of the invention to improve the range of motion (ROM) by having a minor radius pivot point during the second half of the flexion cycle to allow internal femoral rotation thereby avoiding bone impingement.

It is an object of the invention to improve knee strength by better dynamic alignment and mechanical advantage of the quadriceps mechanism by keeping the femoral condyle facing the patella/quadriceps muscle accomplished by the stable pivot (‘Medial Pivot’ or Lateral Pivot’) of external rotational femoral motion during early flexion. This pivot motion creates strong leverage (increase quadriceps moment arm) during walking, stair climbing and sit to stand activities.

It is an object of the invention to provide a knee implant which improves durability of the knee by motion of this design with reducing the existing unstable knee motion of arthritic knees through substituting stable ‘dual pivot’ mechanics which lock the pivoting condyle (either Medial or Lateral) in place while the opposite condyle glides in the desired direction allowing for the needed femoral rotations and thereby lower the articular surface stress which in turn imparts less stresses at the bone-implant interface.

It is object of the invention to provide a knee implant which improves durability of the knee by providing ample knee contact surfaces with congruent stable pivot motions providing low articular surface stresses compatible with the implant's biomaterials during walking, stair climbing and sit to stand activities.

It is an object of the invention to provide a less invasive surgery than currently required for many existing knee designs since surgeons have the surgical options of leaving the patient's remaining ligaments and bone largely intact.

It is an object of the invention to provide a knee implant in which the surgeon can choose the optimal pivot locations (‘Medial Pivot; or ‘Lateral Pivot’) in the knee for the ‘dual pivot’ mechanics (major femoral radius pivot point in early flexion and minor femoral radius point in late flexion.

It is an object of the invention to provide a knee implant which does not require the additional surgical step of a lateral release of the patella since the implant dynamically self-aligns the femoral-patella groove with the natural lateralized position of the patella/quadriceps mechanism which inserts into the tibial tubercle.

It is an object of the invention by ‘dual pivot’ mechanics, to induce the femur to self-align with the patella/quadriceps muscle by the external pivoting motion of the femur occurring during early flexion thereby permitting the patella to smoothly engage the femoral-patellar groove enhancing stability and strength of the knee.

It is an object of the invention to permit the replaced arthritic knee to pivot initially externally harnessing the patella/quadriceps for knee strength in the first half of the flexion cycle, then switch to an internal pivoting motion during later flexion allowing greater flexion with less impingement.

It is an object of the invention to reduce the need for a more constrained implant designs to address different existing arthritic, unstable conditions regardless of cruciate ligament damage; i.e requiring posterior stabilized techniques with a post and cam, a more aggressive surgical technique requiring more bone resection as well as a ‘more constrained’ implant concept.

It is an object of the invention to provide a ‘dual pivot’ knee implant which is designed to take advantage of the properties of new biomaterials such as ultra-high molecular weight polyethylene (UHMWPE), cross linked polyethylene with vitamin E, metallic materials such as cobalt-chromium or titanium, but may be formed from other materials, such as metals, a ceramic material, a polymer material, a bio-engineered material, carbon fiber, nylon, glass, polyethylene, polyester, polytetrafluoroethylene or the like, in other embodiments. These materials may be used alone or in combination with various metals, ceramics, and coatings.

It is an object of the invention to provide a knee implant which can be personalized during implantation to a patient's own anatomy and knee motion: ie the surgeon chooses the optimal dual pivot locations; Medial Pivot’ or ‘Lateral Pivot’ in the knee.

It is an object of the present invention to provide a knee prosthesis specifically designed to provide substantially complete surface-to-surface contact between the congruent articular implant surfaces of a femoral condyle and its reciprocal proximal surface of a tibia throughout a significant range of weight bearing flexion of the knee joint for the lowest implant wear conditions and the longest durability.

It is an object of the present invention to provide a knee prosthesis specifically designed to induce variable pivoting motions about the longitudinal axes of the femur and tibia as the knee flexes during different activities. This is accomplished during walking/running activity by restricting the translational movement (stabilizing) of the knee's femoral component and tibial component; while allowing axial pivoting the major femoral radius in xx the first half of the flexion cycle, then later in the flexion cycle switching to pivoting the minor femoral radius in the other compartment.

It is an object of the invention to provide a knee implant which allow the knee to have stable axial pivoting for weight bearing (high stress) activities of the knee (first half of the knee flexion cycle) during walking and running.

It is an object of the invention to provide a femoral implant design in which both the medial and lateral condyles have various spherical portions or spherical radii that contact one of two tibial implant designs (‘Medial Pivot’ or ‘Lateral Pivot’) having the matching set of reciprocal tibial implant recesses as the knee moves.

It is an object of the invention is to provide a femoral implant design in which the both Medial and Lateral condyles may have a single, distal, major radius of curvature for the arc as flexion moves from −10 degrees (or more) of hyperextension to 75 degrees of flexion (total=85°).

It is an object of the invention to provide a femoral implant design which may have a patellar-femoral groove with a constant coronal radius of curvature to allow maximal surface contact of a highly congruent hemispherical or domed patellar implant.

It is an object of the invention is to provide a femoral implant design which may have a patellar-femoral groove that extends posteriorly between the femoral condyles to maximize surface contact with the patellar implant to at least 100 degrees or more of flexion.

It is an object of the invention to provide the surgeon more flexibility of implantation since the implant has a wide range of acceptable femoral-tibial axial alignments which allows the tibial component to be positioned freely for more optimal bone coverage without limiting axial rotation of the knee (since the implant moves freely in axial motion), thus making this design easier for the surgeon to implant consistently with excellent bone coverage and the desired functional results.

It is an object of the invention is to provide a femoral implant that may be designed to articulate with a traditional ultra-congruent (anterior-posterior lipped) tibial insert, or a low-profile pivoting tibial insert or a mobile bearing (medial & lateral congruent) tibial insert; all of which can assemble into a metal tibial base and may be either stationary or mobile. This flexibility provides the surgeon with significant versatility to treat a variety of patient indications using the same knee system and instrumentation thereby expanding the treatment option intra-operatively for the surgeon, without compromise.

It is an object of the invention is to provide a knee prosthesis that can also be used in conjunction with a ‘cam and post’ mechanism in order to yield a more traditional stabilized design when collateral ligaments are deficient or other major instability exists.

It is an object of the invention to provide a femoral implant with distal medial and lateral condyle possessing a spherical radius that matches the reciprocal radius of the lateral tibial surface, thus creating a stable ‘ball and socket’ type articulation for this compartment during weight bearing activity (the first half of the knee flexion cycle).

It is an object of the invention to provide a tibial implant that has curved ‘half-pipe like’ surfaces which sweeps in an arcuate fashion allowing a femoral condyle to glide freely while maintaining coronal contact with the tibial surface.

It is an object of the invention to provide a tibial implant in which a tibial recess shape allows the smooth knee motion while the knee pivots around the desired ‘Medial Pivot’ or ‘Lateral Pivot’ while sweeping a smooth arc in the opposite compartment during the weight bearing activity of early flexion.

It is an object of the invention to provide a tibial implant which provides a minimum of at least 5 degrees internal and 10 degrees external tibial axial rotation with respect to the femur which is an improvement in function over most existing designs.

It is an object of the invention to provide a tibial implant whose anterior aspect may have a gentle anterior bevel to better accommodate the patellar ligament without impingement.

It is an object of the invention to provide a tibial implant that may have a PCL recess in the posterior aspect to allow the PCL to be easily retained without removing excess bone from its origin.

It is an object of the invention to provide a tibial implant that may have a beveled posterior-lip located at the posterior aspect of the tibial insert which enables greater knee flexion while maintaining stable anterior/posterior translation while avoiding impingement of femoral bone on the tibial component.

It is an object of the invention to provide a tibial implant having maximal tibial surface contact with the femoral surface in either the lateral and medial knee compartments at various parts of the knee flexion cycle, thereby reducing implant stress and wear.

It is an object of the invention to provide a knee prothesis with means to control anterior-posterior translation of the femur on the tibia with ‘ball & socket’ capture of at least one condyle (‘Medial Pivot’ or ‘Lateral Pivot’) during flexion especially during high energy activities making the knee function significantly more stable and stronger.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE SEVERAL VIEWS OF THE DRAWINGS

FIG. 1 is a view of a knee prosthesis.

FIG. 2 is a side view of a femoral component of the knee prosthesis showing four femoral radii (two major and two minor), two (one major+one minor radii) on the medial condyle and two (one major+one minor) on the lateral condyle.

FIG. 3 shows the lateralizing and naturalizing of the femoral patellar groove.

FIG. 4 shows the motion of the medial or lateral condyle (either can be chosen by the operating surgeon to best fit the patient) in its initial tibial ‘pivot point’ or ball-in-socket guiding the femoral external rotation during the first half of knee flexion.

FIG. 5 shows the motion of the medial or lateral femoral condyle in the first 50% of knee flexion guiding the femur to externally pivoting on the tibia and align with the patellar/quadriceps muscle optimizing stability and strength in the knee.

FIG. 6 shows the dual pivot motion of the medial and lateral femoral condyle (either being chosen to initially be at the first tibial pivot point on the tibial plate during complete knee flexion.

FIGS. 7a-c shows the position of the tibia, femur, and patella in full extension (standing) with the present invention and how either medial or lateral femoral condyle can initiate the external femoral pivot on the tibia.

FIGS. 8a-c shows the position of the tibia, femur in early flexion (walking) as the femur externally rotates on the first tibial pivot (either medial or lateral major radii) to capture the patellar/quadriceps muscle leading to stability and maximum knee strength.

FIGS. 9a-c show the position of the tibia, femur, and patella in the present invention when in full flexion (kneeling) engaging the second tibial pivot point (either medial or lateral minor femoral radii) guiding femoral internal rotation for smooth flexion without tibial impingement.

FIG. 10 shows the ‘dual pivoting’ motion of the femur relative to the tibial implant which can be placed on the right or left knee in a reciprocal fashion by the surgeon creating optimal knee stability, strength, and flexion.

FIG. 11 further shows a left or right tibial implant guiding the motion of the femoral implant relative to the tibial implant demonstrating purpose of the trough and pivot points followed by the femoral condyles which initially externally pivot on the tibia during early flexion to align with the patellar/quadriceps muscle, then later femoral internally pivoting on the tibia to avoid boney impingement during high flexion activities.

### DETAILED DESCRIPTION OF THE INVENTION

Most existing knee replacement designs attempt to restore normal function to the damaged, arthritic knee at the cost of mechanical failure of the implant as exemplified by poor motion, poor durability (increased wear), and poor strength. Much of this inadequate function and lack of durability is due to the inability of existing designs to really stabilize abnormal motion pattern of the arthritic knee. These designs, by attempting to force a return to ‘normal’ on these aging knees, create high implant and bone stresses leading to early failures. This invention is the result of significant discoveries through studying pathologic wear patterns of arthritis as well as novel imaging technologies which visualizes and precisely quantify motions inside the living knee (using fluoroscopic, CT and/or MRI) for different disease and injury states. Therefore, embodiments of the invention could be designed with special insight to enhance existing motion patterns into controlled, consistent patterns which properly engage the quadriceps and hamstring muscles while providing a stable articular surface thus avoiding the pitfalls of many present designs.

In the context of the knee, a stable joint is one in which unintended slippage or sliding is limited between the lateral and medial condyles of the femur and their respective lateral and medial tibial surfaces. Some gliding of each condyle within the tibial's curved-trough recess is required due to the pivoting nature of the knee motion. However, the condylar gliding in this design is controlled by always having one condyle anchored at the stable ‘pivot’ point so that the knee experiences no unnecessary, inefficient motion. In this invention, the knee while in the first half of flexion is stabilized at the pivot point which allows external femoral rotation then gradually shifting to internal femoral rotation pivoting at the minor femoral radius as the knee goes to high flexion activities.

Another source of knowledge driving this new invention are reports of carefully documented measurements of knee wear patterns (foot-prints’) in these aging, ligament deficient, and arthritic knees. Observing these ‘foot-prints’ gave insight for this invention to replace the arthritic surfaces of the knee in an extremely efficient and functional manner while restoring the leg's alignment deformity. Also, by maintaining existing ligaments/muscles and using implant surfaces to guide and limit the existing unstable arthritic motion patterns by using the dual pivot mechanism thus improving function for the active patients in walking, stair climbing, kneeling, squatting, etc., (without requiring a ‘cam/post’ implant mechanisms as seen in many existing designs which can create increased wear and stress in the implant).

Embodiments of the invention capitalize on the natural asymmetrical configuration between medial and lateral compartments of the knee. In initial flexion (first half of the flexion cycle about 0-75 degrees), the chosen knee compartment has a generally congruent, spherical contact for an external axial pivot to align the femur with the patella/quadriceps muscle, giving power during the activities of walking and stair or hill climbing, then later, in higher flexion activities such as squatting, lunging or kneeling the axial pivot point shifts to the opposite compartment and utilizes the minor femoral radius for pivoting into internal femoral rotation allowing less impingement and higher flexion.

Embodiments of the invention provide one compartment that is more open and allows the femoral condyle to sweep out an arc similar to a sled traveling down a curved trough, ‘half-pipe’ shape while the other condyle provides the stable pivot point in the other knee compartment. This configuration induces the femoral component to freely rotating externally toward the patella (attached at the tibial tubercle which sits anterolateral on the tibia) leading to early engagement of the quadriceps/patellar mechanism and a positive lock of the patella into its groove (and quadriceps muscle) between the femoral condyles. This stable knee motion allows for quadriceps and hamstring muscles to function at their strongest position as the knee flexes and extends. The implant's stable pivot point at this articular surface prevents slippage and allow patients to return to their desired activities quicker and better after knee replacement. This invention accomplishes all of this under low wear and low stress conditions for this novel implant to have long-term durability.

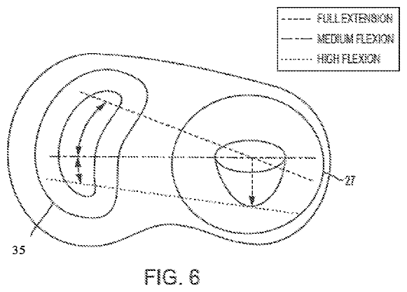

Both femoral condyles have two main radii, a major (distal surface) and a minor (posterior surface) radius with the distal medial condyle having the largest of these condylar radii which contacts the tibia in full extension and through early flexion arc, thus allowing one condyle to have maximum congruency with the other condyle gliding in the ‘half pipe’ recess of the tibial surface. (See FIGS. 6, 9 and 14) The smaller or minor condyle radius comes into play at flexion above 75 degrees (second half of the flexion cycle) and forms the stable second (dual) pivot thereby allowing internal rotation and higher flexion. As noted above, the patella also stays locked into the patella groove giving maximizing stability.

According to embodiments of the invention, the two main radii of the lateral condyle will be smaller than the corresponding two main radii of the medial condyle. This provides optimal surface area (smaller lateral compartment) for maximum congruency in the lateral compartment for axial pivoting, which minimizes or eliminates sliding on the lateral side during early flexion. The larger of the lateral condyle radii is the distal portion of the lateral condyle which has full contact the tibia at extension. The smaller lateral condylar radius is the posterior portion which contacts the tibia at flexion greater than 75 degrees and allows this condyle to escape the full congruency of the lateral hemispherical surface and slide forward in cooperation with the medial pivoting (creating the ability for the tibia to rotate externally) gaining higher flexion.

Embodiments of the invention provide dual pivoting mechanics in the replaced knee aligning and balancing as it flexes during high stress activities of walking and running. Later, during the higher flexion activities, the pivot point shifts to the minor femoral radius and the opposite compartment to provide internal rotation of the femur allowing the femur to internally rotate, thereby avoiding bone impingement and improving range of motion (ROM).

In embodiments of the invention, knee strength is improved by the dynamic alignment and mechanical advantage of the quadriceps mechanism provided by the knee prosthesis. This is accomplished by the variable axial pivoting motion which keeps the patella/quadriceps muscle facing the femoral-patellar groove throughout the range of motion while the contact points move posteriorly, increasing the muscle moment arm, thus creating a very powerful, stable articulation during walking, stair climbing and sit to stand activities.

Embodiments of the invention improves durability and function of the knee by controlling the unstable arthritic knee motion with ‘dual pivot’ mechanics. This design provides increased, congruent knee contact surfaces which are more stable, creating stable motions that reduce the stresses on the articular surface and bone implant interfaces during activities like walking, stair climbing and sit to stand. The design provides complete congruent surface-to-surface pivot point in the knee compartment (upper surface of a tibial component and the condylar surface of a femoral component) throughout early knee flexion during walking and running. This pivot during early flexion causes the femur to externally rotate and face and engage the patella/quadriceps muscle which powers the knee in dynamic weight bearing, high stress activities. Then later (after 75 degrees) the pivot point switches to the minor femoral radius in the opposite compartment allowing internal rotation of the femoral compartment and less impingement, therefore higher flexion.

Embodiments of the invention provide a knee prosthesis specifically designed with variable (DUAL) pivots as the knee moves through full ROM for various activities. This is done by initially externally pivoting in either medial or lateral (surgeon's choice) knee compartments (the first pivot point is for external rotation of the femur on the tibia to guide alignment of patellar/quadriceps muscle for maximum strength), then in later flexion of the knee, the pivot point switches to the other compartment (second pivot point for internal rotation of the femur on the tibia) thus guiding the posterior femoral condyles to move in a posterior direction avoiding impingement with the tibia for high flexion activities like kneeling, squatting or lunging activities.

Embodiments of the invention provide a femoral implant design that may have a patellar groove that is slightly lateralized between the wider medial condyle and the narrower lateral condyle creating this intercondylar groove to guide the patella. The patella starts lateral and proximal to the femoral/patellar groove in full extension, then with flexion and femoral external rotation (first femoral pivot), the patella begins moving posterior and deeper into the groove, harnessing the quadriceps muscle for knee strength. With more flexion, the femur begins internal rotation (with the second femoral pivot point) and the captured patella travels with the femur moving deeper in the groove distally and posteriorly between the femoral condyles keeping the quadriceps muscle fully centered in deep flexion activities (the powerful patella/quadriceps mechanism as the major driver of knee motion).

In embodiments of the invention, the knee moves with an axial pivot points located medial or lateral (surgeon choice of that specific tibial shape) in the first half of the flexion cycle, before switching to a minor femoral radii axial pivot point in the opposite compartment during the latter half of the flexion cycle.

In one embodiment, the right or left femoral implant design has medial and lateral condyles which may have a single radius of curvature from 10° or more of hyperextension to 75° (85° total) or more of the flexion cycle.

In another embodiment, the femoral implant has a lateral condyle which may have one radius of curvature for the first 50% of the range of motion (−10° to) 75° and a second smaller radius of curvature for the second 50% of the range of motion (75° to) 150°.

In one embodiment, the medial condyle may have one radius of curvature for the first 50% of the range of motion (−10° to) 75° and a second smaller radius of curvature for the second 50% of the range of motion (75° to) 150°.

In certain embodiments of the invention, the femoral implant has a patellar femoral groove with a constant radius of curvature in the coronal plane that allows articulation of a highly congruent hemispherical or domed patellar implant. The patellar groove extends posteriorly to enable optimal surface contact area (with lower stresses) for the patellar implant for at least 100 degrees or more of flexion.

The right or left femoral implant may be designed to articulate with either a traditional anterior/posterior lipped (ultra-congruent) ‘cruciate sacrificing’ tibial insert, or a variable pivot (lateral or medial) congruent (PCL sacrificing or sparing) tibial insert, both of which assemble into and are stationary with a metal tibial base. This also may work with a mobile bearing (medial & lateral congruent) design which may include the stable pivoting features. This flexibility provides the surgeon with significant versatility to treat a variety of patient indications with the same knee system and the same instrumentation while easily varying the treatment option intra-operatively, without compromise or the need for more implant inventory and extra instruments.

Embodiments of the invention comprise of a single femoral, patellar and tibial (base plate with insert) implant for left or right knee designs that provides the surgeon with the flexibility to preserve or resect the posterior cruciate ligament (PCL). In the case of resection of the PCL, this knee prosthesis offers the following advantages over a traditional PCL substitution type design (i.e., a design with a traditional cam and post): a) the knee prosthesis is more bone preserving as the bone in the inter-condylar recess is not removed to accommodate an implant housing, b) the patellar groove is further extended posteriorly and lateralized on the femoral component (improving stability and wear characteristics) since no implant housing is required, c) the dislocation safety factor (dsf) is also improved over existing designs (>10 millimeters or more at all angles of flexion).

The tibial implant has the ability to match hemispherical and curved-trough recessed tibial surfaces with four femoral radii (two on each femoral condyle) at various parts of the knee flexion cycle creating the first pivot point for a major femoral radii in early flexion, guiding the external femoral rotation needed to align the femoral-patellar groove with the patellar-quadriceps muscle to provide the early strength and stability during gait and running. Then, during the second half of the flexion cycle, the tibial implant matches the minor femoral radii in the opposite knee compartment, guiding internal rotation of the femur to avoid femoral impingement on the tibia and allow high flexion activities like kneeling and squatting. The surgeon makes the choice of the tibial shape which best suits the patient, providing the best knee function. The surgeon has two possible tibial shapes which match the femoral condylar radii for both right and left knees.

The tibial surface has two types of recesses which are optimized to provide early flexion femoral external rotation and later flexion internal femoral rotation. These are ‘half-pipe’ shaped recesses and hemispherical recesses which match the femoral radii at various parts of the flexion cycle, first pivoting a major radius of the femur into external rotation to align with the patellar/quadriceps muscle, then secondly during the last 50% of flexion, pivoting the opposite minor femoral radius with the tibia's opposite compartment, guiding the femur's internal rotation and allowing deep flexion without femoral impingement on the tibia.

The tibial articular surface optionally has a gentle beveling of the anterior lip to accommodate the patellar ligament in flexion preventing impingement and damage. There may also be PCL recesses both in the medial edge of the posterior lateral femoral condyle as well as the tibial tray to allow the PCL extra freedom of motion.

The surface of the tibial implant has two hemispherical radii and two half-pipe shaped recesses to match with the various femoral condylar radii providing the dual pivot mechanics for the different stages of flexion. This dual pivot mechanism provides the initial external femur rotation (first pivot) to align the patellar-femoral groove with the patellar/quadriceps muscle, then in later flexion guide the internal femoral rotation (second pivot) to allow deep flexion without boney impingement of the knee. tibial impingement of femoral bone posteriorly which occurs when femoral component is positioned to far anterior on the tibial component.

Embodiments of the invention are configured to reduce the need for separate implant designs to accommodate different existing arthritic conditions regardless of cruciate ligament damage; i.e does not require a posterior-stabilized (PS version with post/cam design) implant requiring different surgical tools as well as different implants (more inventory).

The tibial and femoral implants may be made out of any suitable biomaterial having the required mechanical and biological properties. Most preferably they are made from one or more of new biomaterials such as ultra-high molecular weight polyethylene (UHMWPE), cross linked polyethylene with vitamin E, metallic materials such as cobalt-chromium or titanium, but may be formed from other materials, such as metals, a ceramic material, a polymer material, a bio-engineered material, carbon fiber, nylon, glass, polyethylene, polyester, polytetrafluoroethylene or the like, in other embodiments. These materials may be used alone or in combination.

The surgery for implantation is less extensive than required for existing knee designs because the patient's remaining ligaments and bone are left largely intact. The present design also avoids lateral release of the patella as a required surgical step because the knee implant dynamically aligns the femoral patella groove with the natural lateral position of the tibial tubercle and patella/quadriceps mechanism. The present design can be personalized during implantation to a patient's own anatomy and knee motion. The surgeon chooses the pivot locations best driving the initial external rotation of the femur (first pivot point), then the second pivot point follows naturally in the opposite compartment and with the minor femoral radii contacting the tibia in high flexion. The surgeon is provided more flexibility in terms of axial alignment of the knee implants allowing the surgeon more optimal tibial bone coverage without constraining rotational alignment, (freely pivoting) thus making this design easier for the surgeon to implant yet consistently obtain excellent function and results.

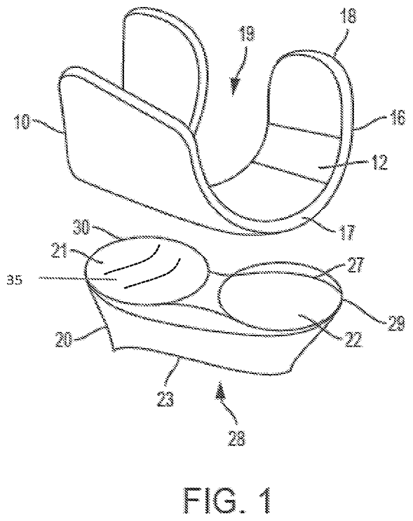

An embodiment of the knee prosthesis 1 is shown in FIGS. 1-11 as described below.

Referring to FIG. 1, a femoral component 10 mounts to the distal end of the femur 12 and a tibial component 20 for mounting to the proximal end of the tibia 21 for articulating with the femoral component.

The femoral component 11 includes a lateral condyle portion 13, a medial condyle portion 14 and a patellar flange portion 15 which joins the anterior ends of the lateral and medial portions 13, 14. The lateral and medial condyle portions 13, 14 being substantially parallel and spaced apart from each other for form a recess 19. Each of the lateral and medial condyle components 13, 14 has an outer surface 16 for articulating with the tibial component 20.

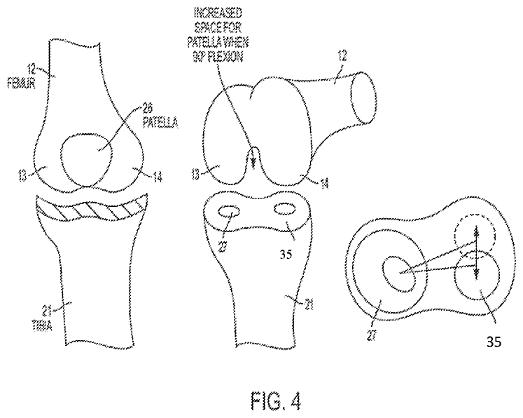

The outer surface 16 of each condylar portion 13, 14 preferably has a distal portion 17 for articulating and engaging a portion of the tibial component 20 when the knee joint is extended and partially flexed as shown in FIG. 4, and a posterior portion 18 for articulating and engaging a portion of the tibial component 20 when the knee joint is flexed substantially 90 degrees as shown in FIG. 4.

The femoral component 11 may include typical attachment aids for helping to secure the femoral component 11 to the distal end 10 of the femur 12. Such attachment aids may include one or more pegs, fins, surface treatments, etc., as will now be apparent to those skilled in the art.

The femoral component 11 may be constructed in various manners and out of various materials as will now be apparent to those skilled in the art. Thus, for example, the femoral component 11 can be machined, cast, forged, or otherwise constructed as a one-piece, integral unit out of a medical grade, physiologically acceptable metal such as a cobalt chromium alloy or the like, in various sizes to fit a range of typical patients, or may be custom-designed for a specific patient based on data provided by a surgeon after physical and radiography examination of the specific patient, etc.

The tibial component 20 includes a base or tray member 23 for being secured to the proximal end 24 of the tibia 25. The tibial component 20 can be constructed so that it is directly attachable to the proximal end of the tibia 24 or it may be designed to mount to a separate base plate, not shown.

The tibial component 20 preferably includes attachment aids for helping to secure it to the proximal end 21 of the tibia 19. Such attachment aids may include one or more pegs, fins, screws, surface treatments, etc., on the lower surface of the tibial component thereof as should be readily apparent to one skilled in the art. In one such embodiment a peg 26 is used as shown in FIG. 8.

The tibial component 20 may be constructed in various manners and out of various materials as will now be apparent to those skilled in the art. Thus, for example, it can be machined or otherwise constructed as a one-piece, integral unit out of a medical grade, physiologically acceptable metal such as a cobalt chromium alloy or the like, in various sizes to fit a range of typical patients, or may be custom-designed for a specific patient based on data provided by a surgeon after physical and radiography examination of the specific patient, etc. The upper surfaces may have various finishes applied including highly polished for load bearing surfaces.

The tibial component 20 has an upper bearing surface 27, a lower or distal surface 28, a medial side 29, a lateral side 30, an anterior or front side 31, and a posterior or rear side 32. The lower surface 28 is designed for mating to the tibia 25 or to a base plate. The sides 28, 29, 30 and 31 of the tibial component 20 preferably has a generally angle upward and outward from the lower surface 28 to the upper surface 27 thereof.

There are two configurations of the upper bearing tibial surface which can be chosen by the surgeon and each generally has two hemispherical recesses and two curved-trough recesses; each for a major femoral radius and for a minor femoral radius. Both of these tibial configurations provide dual pivoting motions at the knee with both provide external femoral rotation (first pivot-major radius) in the first half of knee flexion (aligning the femoral-patellar groove with the patellar/quadriceps muscle), then providing internal femoral rotation (second pivot-minor radius) during the second half of knee flexion which avoids boney impingement and allows high flexion activities. One or the other of these tibial configurations can be implanted to optimize these rotations in a particular patient. Medial or lateral compartment a 21 for pivotally receiving and coacting with the outer surface 16 of the major condyle portion 13 of the femoral component 11, and a medial compartment 22 for pivotally receiving and coacting with the face surface 16 of the medial condylar portion 14 of the femoral component 11. The medial or lateral compartment 21 can be retained in the condyle portion 13 in a ball and socket type of arrangement in early flexion for the first pivot (major radius). Thus, medial or lateral compartment 22 is chosen by the surgeon for the initial major radius first pivot into external femoral rotation while shaped to receive the other condyle portion 14, is elongated such that this condyle portion 14 can glide in this compartment 14 completing the needed femoral rotation motion during the particular portion of the knee cycle. The medial and lateral compartments are sufficiently congruent to allow adequate surface contact with the medial and lateral condyle portions for proper support and low wear characteristics.

Each tibial implant 20 includes both congruency and controlled gliding for restricting the slid-able movement of the articular bearing member 45 relative to the base member 43 but allows a swinging motion in the medial portion of the upper surface 67 of the articular bearing member 45 with pivotal motion of the condylar portion of the upper surface 67 of the articular bearing member during early flexion 45. The restriction means may be designed to restrict the pivoting motion of the articular bearing member 45 to approximately 20 degrees of internal/external rotation of the knee joint 13 as will now be apparent to those skilled in the art. The restriction means may be constructed or formed by various structures.

One of skill in the art will appreciate that the medial and lateral condyle portions 13, 14 can be placed as recesses on the tibial component 22 to enhance the particular, needed femoral rotations, first for important muscle alignment during high load activities, then next for important avoidance of boney impingements during high flexion activities. The convex bearing surfaces can be placed on the femoral component 11.

The method of replacing a knee joint using the joint prosthesis 1 typically starts with standard preoperative planning to estimate the size of the prosthesis to be implanted. The knee joint can then be exposed in any typical manner. The distal end 10 of the femur 12 and the proximal end 24 of the tibia 25 can then be resected and prepared, and a trial reduction of the knee joint with optimal tibial surface (one of two choices) being chosen by the surgeon that first allow the best external femoral rotation in early flexion, then with maximal knee flexion, guide optimal internal femoral rotation performed using appropriate trial implants. Upon successful trial reduction, an appropriate femoral component 11 is implanted on the prepared distal end 10 of the femur 12, and an appropriate tibial component is implanted on the prepared proximal end 24 of the tibia 25. After final testing for motion and stability, the surgical site can be closed in any typical manner.

Using embodiments of the invention, the posterior cruciate ligament (PCL) 41 can be readily retained to provide more stability along with the medial collateral ligament 42 (MCL) and lateral collateral ligament (LCL) 43.

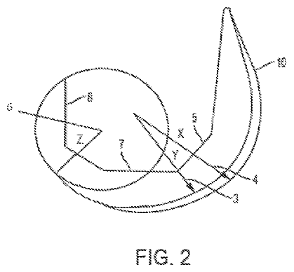

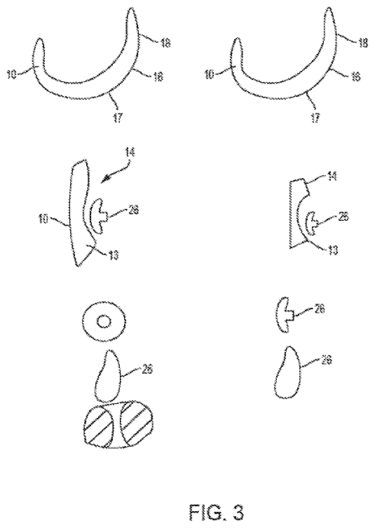

Referring to FIGS. 2 and 3, the width of the lateral condyle 13 is smaller than the width of the medial condyle 14 thereby shifting the patellar groove laterally. This is more natural for the laterally positioned patella thus providing increased stability and lower stress for the femoral-patellar joint.

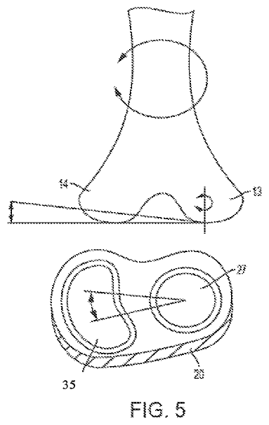

Referring to FIG. 4, which show the position of the patella 26 in the patella groove 19 during extension and flexion. The sliding motion of the medial condyle 14 during the first 75 of flexion is shown in FIG. 5.

Referring to FIG. 5, the differences in motion of a knee can be observed. In extension, the lateral and medial condyle portions 13, 14 of the femoral component 11 are in full contact with the lateral and medial concavities 27, 28.

In early flexion, the femoral component major radii 11 pivots in the matching tibial hemispherical recess rolling out externally (first pivot point) to align with the patella 26 and the quadriceps 40 while the opposite condyle portion 14, glides along the tibial ‘half-pipe’ surface 28. For the first 75 degrees or so of motion, the major radii of the femoral condyle 11 and the tibial hemispherical surface 20 are rotating congruently with full contact. Beginning at about 75 degrees the tibial and femoral components begin to lose congruency with the motion becoming a sliding motion allowing translational motion to occur with the axial pivot point shifting to the second pivot into internal femoral rotation.

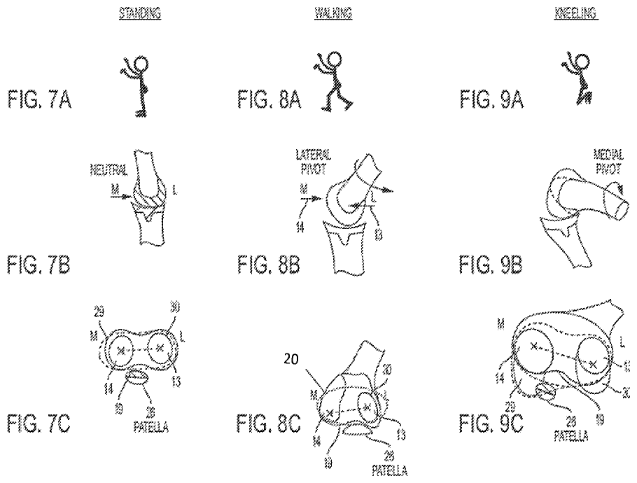

Referring to FIGS. 7a-c that show a relatively equal and centered weight distribution between the medial condyles 14 and the lateral condyles 13 on the medial compartment 29 and the lateral compartment 30 while a person is standing without movement. When standing the patella 26 is centered in the patellar groove 19.

Referring to FIGS. 8a-c which shows the first pivot while a person is engaged in walking activity, the patella 26 and its femoral groove align accomplished by this external femoral rotation created as the knee pivots laterally through a range of motion from about −10° to about 75°.

Referring to FIGS. 9a-c which shows the knee in the second pivot into internal rotation at a range of motion over 75° as might be observed in kneeling or squatting. At this level of flexion, the minor condyle radius 13 becomes the axial pivot point (second pivot) on the posterior tibial hemispherical surface in this compartment 29 and the patella travels in the deepened portion of the femoral-patellar groove 19.

In all ranges of motion, the patella remains locked in the patellar groove (stronger locking with more flexion) 19.

A knee prosthesis for replacing at least a portion of a knee joint between the distal end of a femur and the proximal end of a tibia, the knee prosthesis comprising a femoral component for mounting to the distal end of a femur comprising a lateral and a medial condyle, each condyle having a major (first) and minor (second) radius (a total of four condylar radii: two medially & two laterally on the femoral component). The major radii of both condyles on the femoral implant are located on its distal aspect while the two minor radii are located posteriorly on each condyle. A tibial component for mounting to the proximal end of a tibia; said tibial component having four recessed areas in its proximal (articulating) surface each corresponding to matching femoral radius contacting or articulating with said tibial component at particular times in the knee flexion cycle. Two of the tibial component recesses are approximately hemispherical and serve as one of the two pivot points (Dual Pivot Mechanics) to be chosen by the surgeon. The other two tibial component recesses are curved-troughs shapes providing tracks for the non-stationary condyle to glide in either external rotation (first pivot) or internal rotation (second pivot).

At less than 75 degrees of flexion, the major femoral radii of both lateral and medial femoral condyles are in contact with the lateral and medial tibial recessed surfaces. The surgeon will choose the tibial implant which has one of these major condyle radii becoming the stable pivot point (medial or lateral) as the other becomes the gliding condyle, initially produce the needed external femoral rotation which aligns the femoral-patellar groove with the patellar/quadriceps muscle. This tibial implant will have in its opposite compartment the second stable pivot point of the minor femoral radius which engages the matching tibial recess in higher knee flexion guiding the internal femoral rotation which avoids boney impingement and allows higher flexion activities. Thus, a surgeon will have two choices of tibial implants for each right or left femoral implant. One having the initial (major radii) pivot point medial and the other having this initial pivot point lateral, both producing external femoral rotation, but with different pivoting mechanics. The minor radii pivot point will follow in the opposite knee compartment, producing the necessary internal femoral rotation. Each of these two tibial implant selections interact slightly differently with the existing knee muscles and ligaments allowing the surgeon to optimize the desired knee performance.

At flexion greater than about 75 degrees, the second (minor) radius of the lateral femoral condyle now begins to translate forward in the anterior lateral recess while the medial femoral condyle translates posteriorly and assumes maximal congruency with the second (minor) medial condyle radius thereby allowing the medial condyle to assume the pivot point during this second half of the flexion cycle creating the internal femoral rotation required for high flexion activities.

In some instances, during the first half of the knee flexion cycle, the articular contact (pivot) point of the knee is centered about a point located generally over the posterior one third of said lateral proximal surface of said tibial component; wherein this articular contact (pivot) point shifts to the posterior surface of the medial knee compartment during the second half of the knee flexion cycle.

In all instances the invention prosthesis allows the femoral implant to initially pivot externally on the surface of the tibial component and align the femoral-patellar groove with the quadriceps muscle complex allowing for the capture of the patella which dynamically stabilizing all three components of the knee.

Referring to FIGS. 10 and 11, in another embodiment the knee prosthesis for replacing at least a portion of a knee joint between the distal end of a femur and the proximal end of a tibia; the knee prosthesis comprises: a tibial component for mounting to the proximal end of the tibia, said tibial component including a proximal surface having hemispherical surfaces and curved-trough recesses and a femoral component for mounting to the distal end of the femur, said femoral component including a lateral and medial condyle each with a major and minor spherical radius for its spherical surfaces. These four spherical radii pivotally coact with the reciprocal medial and lateral recesses in the proximal tibial surface in accordance with claim 1, said hemispherical posterior recess of the lateral surface of said tibial component, and a medial femoral condylar spherical surface whose major radius movably coacts with said medial surface of said tibial component; the minor radius of the said lateral condylar surface located in its posterior portion allows more translation along said lateral tibial surface during the second half of the flexion cycle as pivoting occurs in the medial compartment coacting the minor radius of medial condyle with the corresponding medial tibial surface. There is a distal side, an anterior side, and a posterior side with a spherical shape only from a posterior point on said posterior side of said face surface of said lateral condylar portion to an anterior point on said anterior side of said face surface of said lateral condylar portion located in an arc (radian) of about 85° from said posterior point; said face surface of said lateral condylar portion having a posterior side, a distal side, an anterior side, and a spherical shape; said spherical shape of said face surface of said lateral condylar portion being substantially congruent with said hemispherical lateral surface of said tibial component so that substantially complete congruent surface-to-surface contact between said hemispherical lateral surface of said tibial component and said spherical face surface of said lateral condylar portion of said femoral component is provided throughout the first half of the flexion cycle (of about 85° (−10° to)) 75° of the knee joint; wherein the knee laterally pivots in the first half of the flexion cycle (from about −10° to about) 75° and pivots medially during the second half of the flexion cycle (from about 75° to about) 150°. A knee prosthesis in which the variable pivot point of the knee is initially in the lateral knee compartment between the lateral spherical condylar portion (major radius) of the femoral component and lateral hemispherical portion of the tibial component before switching to pivoting in the medial compartment between the spherical (minor radius) of the medial femoral condyle with the posterior hemispherical surface of the medial tibial component during later knee flexion.

In some embodiments, the medial condyle portion (major radius) of the femoral component articulates smoothly on the medial sweeping (half-pipe shaped) surface of the tibial component during the first half of the knee flexion cycle.

In another embodiment, the knee prosthesis for replacing at least a portion of a knee joint between the distal end of a femur and the proximal end of a tibia; the knee prosthesis comprising: a tibial component for mounting to the proximal end of the tibia, said tibial component including a proximal surface having a hemispherical lateral surface and a medial surface; and a femoral component for mounting to the distal end of the femur, said femoral component including a first radius portion of a spherical lateral condylar whose face surface pivotally coacts with said hemispherical lateral surface of said tibial component during the first half of the flexion cycle. This lateral condylar portion having a spherical face surface for movably coacting with said hemispherical lateral tibial surface; and being substantially congruent with said hemispherical lateral surface of said tibial component so that substantially complete congruent surface-to-surface contact in this lateral knee compartment between said hemispherical lateral surface of said tibial component and said major radius spherical face surface of said lateral condyle of the said femoral component whose articulation occurs throughout the first half of the knee flexion cycle (from about −10° to) 75° before shifting the pivot point more medial during later knee flexion.

In another embodiment, the knee prosthesis for replacing at least a portion of a knee joint between the distal end of a femur and the proximal end of a tibia; the knee prosthesis comprises:
1. a tibial component for mounting to the proximal end of the tibia, said tibial component including a proximal aspect having a hemispherical lateral surface posteriorly for articulating (pivoting) in the lateral knee compartment during the first half of the flexion cycle; and
2. a femoral component for mounting to the distal end of the femur, said femoral component including two sphere-like condyles (medial and lateral) each with two (one major and one minor) radii on their spherical face surfaces; wherein the medial and lateral condylar portions of the invention are designed to pivotally articulate with the tibial component in a variable fashion; first (during the first half of the knee flexion cycle) on the posterior-lateral portion of this tibial component; then later (the second half of the knee flexion cycle) on the posterior-medial portion of this tibial component; wherein as the pivoting articulation occurs in one compartment at a time, therefore in early flexion, there is anterior translation of the medial condyle while in the lateral compartment there is smooth axial pivoting rotation as the knee flexes allowing the necessary engagement of muscles and ligaments in a coordinated, efficient and powerful fashion. These reciprocal hemispherical surfaces of said tibial component with said femoral component allow for congruent articular contact with low stress and low wear between components. Hence the lateral condylar portion has a spherical face surface for movably coacting with said lateral hemispherical surface compartment of said tibial component; this spherical face surface of said lateral femoral condylar having a major radius with spherical shape from about −10° distal to about 75° posterior then transitioning to a minor radius from 75° to about 150°; said major radius portion of the spherical face surface of said lateral femoral condylar being substantially congruent with said hemispherical lateral surface of said tibial component so that substantially complete congruent surface-to-surface contact occurs in said lateral compartment of the knee during the first half of the flexion cycle, thereafter shifting to less congruency in said lateral compartment when the minor radius of the lateral femoral condyle engages the tibial surface, thus allowing smooth anterior translation of this lateral femoral condyle on the tibial component as the axial pivot point shifts medially during the second half of the knee flexion cycle.

Devices of the present invention can be customized using measurements and other data from imaging devices such as a CT scan or MRI. The dimensions of a patient's knee obtained by imaging the knee can be used to size or custom fit the devices of the present invention. The dimensions of the condyles and compartments can be adjusted to better conform to a patient's current geometry or adjusted to correct deficiencies in the patient's current knee geometry. Sizing or customizing a knee using imaging techniques are known in the art and taught in U.S. Pat. No. 8,974,539. Intra-operative virtual and enhanced reality utilizing artificial intelligence to compile and select best data points may also be used.

One of skill in the art will appreciate that the dimensions and radii disclosed herein can be varied as long as the resulting joint can transition from a lateral pivot to a medial pivot above about 75° of flexion and the patella is retained in the patellar groove.

What is claimed is:

1. A dual pivot right or left knee prosthesis comprising:
(a) a femoral component configured for mounting to a distal end of a right or left femur, said femoral component comprising a lateral condyle and a medial condyle, each condyle having a major radius and a minor radius located posteriorly on each condyle for a total of four condylar radii on a right or left femoral component;
wherein the major radii of both condyles on the right or left femoral component are located on a distal surface of the condyles with the major radius of the lateral condyle having a radius that is equal to or up to 30 percent smaller than the major radius in the medial condyle,
wherein the minor radii are located posteriorly on a right or left femoral component with the minor radius of the lateral condyle having a radius which is the same as or smaller than the minor radius on the medial condyle;
(b) two tibial configurations for a right or left tibial plate having medial and lateral sides, said tibial plate comprising four tibial plate recesses corresponding to the four femoral condylar radii, each of said four tibial plate recesses lying medially and laterally, with two of said four tibial plate recesses being hemispherical and two being trough-like or curved, half-pipe shape, one hemispherical and one trough-like or curved, half-pipe shape recess are located on each of the medial and lateral aspect of the tibial plate; wherein the tibial plate recesses are constructed and arranged to provide a lateral or medial stable femoral pivot point while gliding in an opposite compartment, thereby providing external knee rotation in early flexion allowing the femur to eternally rotate, engaging patella/quadriceps power in early flexion, and then internal knee rotation in late flexion, a second pivot point occurring during deep knee flexion guiding internal rotation motion of the femur on the tibia which avoids boney impingement and allows deep knee flexion,
wherein, if the medial side is the stable major radii pivot point to begin flexion, the major radius of the medial femoral condyle fits into the medial hemispherical tibial recess during early knee flexion starting the external rotation pivoting and causing the lateral femoral condyle major radius to glide posteriorly in the lateral curved trough-like tibial recess;
wherein if the stable pivot point to begin knee flexion is chosen on the lateral side, then the major radius of the lateral femoral condyle fits into the lateral tibial hemispherical recess to begin the external rotation motion of the knee causing the major radius of the medial femoral condyle to glide anteriorly in the medial tibial trough-like recess, thereby during early flexion, the medial or lateral pivot location of the knee is customized to a particular knee with either medial or lateral axial pivoting in a first half of a knee flexion cycle.

2. The knee prosthesis according to claim 1, wherein the femoral condyle maintains congruent surface-to-surface contact with the hemispherical recess of the tibial plate throughout the first half of knee flexion, guiding femoral external rotation and allowing the femoral-patellar groove to engage the patella/quadriceps power in early flexion.

3. The knee prosthesis according to claim 1, wherein the major radius of the femoral condyle of the femoral component is configured to glide smoothly on the curved trough-like portion of the tibial plate, guiding femoral external rotation during the first half of the knee flexion cycle.

4. The knee prosthesis according to claim 1, wherein during the first half of the knee flexion cycle, the stable major radius pivot point (first pivot) of the knee is centered in either the medial or lateral knee compartment causing external rotation and allowing the femur to engage the patella/quadriceps muscle power in early flexion for said high load activities; wherein the minor radius pivot point (second pivot) shifts to the opposite knee compartment during the second half of the knee flexion cycle, guiding femoral internal rotation of the knee needed to avoid boney impingement for high flexion activities.

5. The knee prosthesis according to claim 1, wherein the prosthesis is configured to allow the femoral component to initially pivot externally on the tibial plate, aligning the femoral-patellar groove with the quadriceps muscle complex allowing for the patella to be captured to dynamically stabilize the knee.

6. The knee prosthesis according to claim 4, wherein the high flexion activities are kneeling, lunging, and squatting.

7. The knee prosthesis according to claim 1, wherein early knee flexion is the first 50% of knee flexion.

8. The knee prosthesis according to claim 1, wherein, dual pivot motion of the knee begins with the first stable pivot point (major radii) of the knee in early flexion and begins on the medial or lateral side between the major spherical radius of the femoral implant and the hemispherical recess of the tibial implant causing external femoral rotation, allowing the femur to engage the patella/quadriceps power in early flexion; then switching to a second pivot point (minor radii) during the last half of the knee flexion cycle which occurs in the opposite compartment from the compartment where the first major radius pivot begins, guiding internal rotation of the femur on the tibia preventing boney impingement and allowing high flexion knee activities.

9. A method of stabilizing a knee comprising: mounting a prosthesis for replacing at least a portion of a right or left knee joint between the distal end of a femur and the proximal end of a tibia wherein the prosthesis comprises:
(a) a right or left femoral implant for mounting to a distal end of a right or left femur comprising a lateral condyle and a medial condyle, each condyle having a major radius and a minor radius for a total of four condylar radii on the femoral component; wherein
1. the major radii (distal) of the femoral condyles on the right or left femoral implant are located at the distal surface of the medial and lateral condyles with the major radius of the lateral condyle having a major radius that is equal to or up to 30 percent smaller than the corresponding major radius in the medial condyle; and
2. the minor radii are located posteriorly on the medial and lateral condyles of the right or left femoral implant with the minor radius of the lateral condyle having a radius which is equal to or up to 30 percent smaller than a corresponding minor radius on the medical condyle; and
(b) a right or left knee tibial plate having medial and lateral sides each with two recesses (total of four tibial recesses), comprising;
1. two tibial implant hemispherical recesses correspond to the major and minor femoral radii, wherein a position of each recess on the tibial implant is for a right or left knee and starts either medial or lateral, thus providing two tibial configurations for each left or right knee depending on the chosen location of the first pivot (major radius) point, either medial or lateral knee compartment, wherein for early flexion, the femur rotates externally on the tibia to align the femur with the patellar/quadriceps muscle; wherein if the first pivot point is medial, then the tibial shape will be made according to this configuration or if the first pivot point is lateral, the tibial shape will be according to that pivot location accommodating this first pivot motion with the non-pivoting femoral condyle which glides in the curved trough in the opposite compartment) guiding this initial external femoral rotation.
2. the two curved trough tibial recesses forming a curved half-pipe shape for the smooth gliding of the non-pivoting femoral condyle, thus providing smooth translation while rotating around the stable pivot point initially guiding external rotation of the femur on the tibia during the first half of the knee flexion cycle and aligning the patellar/quadriceps muscle for maximal knee stability and strength, and then a second pivot point utilizing the minor femoral radii is created in the opposite knee compartment from the first pivot location with the minor radius of the opposite femoral condyle, guiding internal rotation of the femur which avoids boney impingment during the second half of the knee flexion cycle.

10. A method of stabilizing a right or left knee comprising: mounting the prosthesis for replacing at least the portion of the knee joint according to claim 9 between a distal end of a femur and a proximal end of the tibia, wherein the femoral condyles maintains congruent surface-to-surface contact with each tibial implant recess (first and second pivot points) throughout knee flexion guiding first external femoral rotation (first pivot point), allowing the femur to engage the patella/quadriceps power in early flexion, then internal femoral rotation (second pivot point) for allowing high flexion without boney impingement of the femur on the tibia.

11. The method according to claim 10, wherein the femoral component and the tibial plate have a dual pivot motion with external rotation during the first half of the knee flexion cycle (first pivot-major radii) and then internal rotation during the second half of the knee flexion cycle (second pivot-minor radii); wherein as the pivoting articulation occurs in one compartment, there is sliding of the other condyle in the other compartment creating a smooth axial rotations first externally then later internally as the knee fully flexes thereby allowing the necessary engagement of muscles and ligaments and avoid boney impingements in a coordinated, efficient and powerful fashion.

12. The method of according to 11, wherein the medial and lateral condyles have spherical condylar surfaces for stable dual pivoting with matching recesses of the tibial plate, wherein the positions of the recesses are determined placement on a right knee or a left knee and by preference and patient's particular anatomy for the first pivot being either in the medial or lateral knee compartment, the spherical face of both femoral condyles having a major radii distally and the minor radii posteriorly on the femoral condyles; wherein the major and minor radii of the femoral condyles are congruent with the matching tibial plate recesses so that complete congruent surface-to-surface contact occurs in the appropriate compartment of the knee during the first half of the flexion cycle guiding the needed external rotation allowing the femur to engage the patella/quadriceps power in early flexion; thereafter during late flexion, shifting congruency to the opposite compartment where the minor radius of the femoral condyle fits congruently into the matching tibial hemispherical surface, thereby allowing smooth internal knee rotation completing the dual pivot motion needed for full knee flexion.

13. A knee prosthesis for replacing a portion of a right or left knee joint between a distal end of a femur and a proximal end of a tibia, the femoral prosthesis comes in reciprocal right and left configurations comprising:
a femoral implants for mounting to the distal end of a right or left femur each comprises a lateral condyle and a medial condyle, each condyle having a major radius and minor radius for a total of four condylar radii on the femoral component; wherein, the major radii of both condyles on the femoral component are located on a distal surface of the condyles and the minor radii located on the posterior aspect of the condyles; the lateral femoral condyle having a major radius that is equal to or up to 30 percent smaller than a major radius in the medial condyle, and the minor radii, located posteriorly on the femoral implant having a minor radius which is equal to or up to 30 percent smaller than a minor radius on the medial condyle;
a tibial implant having medial and lateral aspects, asymmetrically shaped to fit the natural proximal tibia, two hemispherical recesses and two trough-like or curved half-pipe shaped recesses running anterior and posterior and corresponding to particular aspects of the lateral and medial condyles of the femoral implant which contact the tibia as the femoral implant flexes and rotates externally and internally, wherein the tibial implant is configured for mounting to a proximal end of the tibia.

14. The knee prosthesis according to claim 1, wherein said higher load activities are walking or running.

15. A method of stabilizing a right or left knee comprising: mounting a prosthesis for replacing at least the portion of the knee joint according to claim 1 between a distal end of a femur and a proximal end of the tibia.

16. A method of stabilizing a knee comprising: mounting a prosthesis for replacing at least the portion of a right or left knee joint according to claim 13 between a distal end of a femur and a proximal end of the tibia.
